(19) United States
(12) Patent Application Publication
(10) Pub. No.: US 2004/0228821 A1
(43) Pub. Date: Nov. 18, 2004

(54) Personal care products comprising active agents in a gel network

(75) Inventors: Jorge Max Sunkel, Cincinnati, OH (US); Curtis Boddy Motley, West Chester, OH (US); Thomas Elliot Rabe, Baltimore, MD (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/841,194

(22) Filed: May 7, 2004

Related U.S. Application Data
(60) Provisional application No. 60/471,261, filed on May 16, 2003.

(51) Int. Cl. A61K 7/06; A61K 7/11

(52) U.S. Cl. 424/70.12

(57) ABSTRACT

The present invention relates to a personal care product containing a polymeric gel network produced by the process of crosslinking an organopolysiloxane polymer and swelling the organopolysiloxane polymer to form a gel network by introducing liquids including one or more active agents, where the active agents are entrapped in the gel network. The liquids of the product may optionally include one or more solvents.

### CROSS REFERENCE TO RELATED APPLICATION(S)

This application claims the benefit of Provisional Application Serial No. 60/471,261, filed May 16, 2003.

### FIELD OF INVENTION

The present invention relates to personal care products suitable for use on mammalian skin and hair. These products comprise a polymeric gel network that includes an organopolysiloxane polymer and a liquid where the liquid is an active agent or an active agent in combination with a solvent. These products are intended to increase the level of active agent delivered through a personal care composition with pleasant sensory and aesthetic attributes. Further, the products add formulation flexibility and stability to the personal care compositions.

### BACKGROUND

Gel compositions of crosslinked organopolysiloxane polymers swollen in fluids such as dimethicone, cyclomethicone, isododecane, mineral oil, etc. are known in the art (U.S. Pat. No. 6,531,540 B1; U.S. Pat. No. 6,538,061 B2; U.S. Pat. No. 6,444,745 B1; U.S. Pat. No. 6,346,583 B1). These silicone elastomers or silicone gels are useful in the personal and beauty care products arena because they offer both consumer and formulation benefits. They provide pleasant sensory and shine control benefits for the consumer. Further, these materials serve as thickening agents for lotions, creams foundations, mascara, etc., and as suspending and stabilizing agents for emulsions and particulates.

The manufacturing process of these gels consists mainly of two parts. The first part is the reaction among the organopolysiloxane polymers to form a three-dimensional crosslinked polymer network, resulting typically in a powdery or rubbery substance. The second part is the formation of a gel system by swelling the crosslinked polymers with a fluid with the aid of shearing and mixing forces. The final product is a visco-elastic gel of the swollen crosslinked polymer in the fluid that can vary in polymer and fluid content from manufacturer to manufacturer. The properties and in-product performance of the gel network are dependant on the starting polymer molecular weight, reaction chemistry, fluid type, processing forces, and other parameters chosen by the manufacturer to achieve a desired gel performance.

The inclusion of benefit active agents during the crosslinking reaction process, the first part of the process, is known in the art. The addition of actives during the first stage of the reaction has been discussed in U.S. Pat. No. 6,207,717 B1; EP 1020494A1; and EP 1057872A1.

A need still exists for an improved process that allows for the delivery of higher concentrations of actives for greater potency and longer-lasting benefits while creating more formulation space for the inclusion of other personal and beauty care components to be delivered from both a stable and sensory pleasant system. Further, a need still exists for a finished gel product that provides consumer and formulation benefits in a cost-effective manner, delivered from a gel system that is easy for the formulator to employ when in the presence of the additional components of the personal care product.

The inventors of the present invention have surprisingly discovered that the addition of personal care active agents, alone or in combination with other fluids, during the second process step of swelling the crosslinked polymers creates additional formulation flexibility and consumer sensory benefits. Unexpectedly, this process allows for formulation and consumer benefits that can be achieved in a manner that produces a cost-effective gel while preventing a potentially reaction-inhibiting active agent from interfering with the crosslinking of the polymers during the first process step.

### SUMMARY OF THE INVENTION

The present invention relates to a personal care product containing a gel polymeric network produced by the process of crosslinking an organopolysiloxane polymer and swelling the organopolysiloxane polymer to form a gel network by introducing liquids including one or more active agents where the active agents are entrapped in the gel network.

The liquids in the products of the present invention may additionally include one or more solvents.

### DETAILED DESCRIPTION OF THE INVENTION

As used herein, “personal care products” means any color cosmetic, hair, or skin care product. “Personal care composition” refers to the formulation included in the personal care product that confers the desired benefit. “Personal care products” are those used to treat or care for, or somehow moisturize, improve, or clean the skin or hair. Additional products contemplated by the phrase “personal care products” include, but are not limited to, sunscreens, adhesives, bandages, toothpaste, anhydrous occlusive moisturizers, antiperspirants, deodorants, personal cleansing products, powder laundry detergent, fabric softener towels, occlusive drug delivery patches, nail polish, powders, tissues, wipes, hair conditioners-anhydrous, shaving creams, and the like. The term “cosmetic” or “make-up” refers to products that leave color on the face, including foundation, blacks and browns, i.e., mascara, concealers, eye liners, brow colors, eye shadows, blushers, lip sticks, lip balms, face powders, solid emulsion compact, and so forth. The term “foundation” refers to liquid, creme, mousse, pancake, compact, concealer or like product created or reintroduced by cosmetic companies to even out the overall coloring of the skin.

The term “ambient conditions” as used herein refers to surrounding conditions under about one atmosphere of pressure, at about 50% relative humidity, and at about 25° C., unless otherwise specified.

Herein, “comprising” means that other steps and other ingredients which do not affect the end result can be added. This term encompasses the terms “consisting of” and “consisting essentially of”. The products, compositions, and methods/processes of the present invention can comprise, consist of, and consist essentially of the essential elements and limitations of the invention described herein, as well as any of the additional or optional ingredients, components, steps, or limitations described herein.

All percentages, parts, and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

The preferred pH range for the personal care product is from about pH 3 to about pH 10, preferably from about pH 4 to about pH 9.

All measurements made are at 25° C., unless otherwise designated.

I. The Composition

A. Polymer

The composition of the present invention includes a polymer that is linear in nature, non-linear in nature, or a combination thereof. The polymer can be made up of a variety of monomers so long as the polymer is amenable to being swollen with a liquid, mixture of liquids, or a solution comprising a solid dissolved in a liquid. Suitable polymers for inclusion in the claimed compositions include, but are not limited to polysiloxanes that are crosslinked organopolysiloxane polymer gel networks. For instance, particularly well-suited crosslinked organopolysiloxane polymer gel networks are formed from polymerization of an epoxy functional organosiloxane in the presence of an acid catalyst. The organopolysiloxane polymer is a crosslinked organopolysiloxane polymer gel network selected from non-emulsifying polymer gel networks, emulsifying polymer gel networks, and combinations thereof. Specific examples of such are described in U.S. Pat. No. 6,531,540 B1, U.S. Pat. No. 6,538,061 B2, U.S. Pat. No. 6,444,745 B1, U.S. Pat. No. 6,346,583 B1, U.S. Pat. No. 5,654,362, U.S. Pat. No. 5,811,487, U.S. Pat. No. 5,880,210, U.S. Pat. No. 5,889,108, U.S. Pat. No. 5,929,164, U.S. Pat. No. 5,948,855, U.S. Pat. No. 5,969,035, U.S. Pat. No. 5,977,280, U.S. Pat. No. 6,080,394, U.S. Pat. No. 6,168,782, U.S. Pat. No. 6,177,071, U.S. Pat. No. 6,200,581, U.S. Pat. No. 6,207,717, U.S. Pat. No. 6,221,927, U.S. Pat. No. 6,221,979, U.S. Pat. No. 6,238,657, and U.S. Pat. No. 4,987,169. The polymer is present in an amount of from about 0.1% to about 40%, preferably from about 1% to about 30%, by weight of the composition. In preferred embodiments, the polymer is present in an amount of from about 2% to about 20%, by weight of the composition.

B. Active Agents

The compositions of the present invention comprise one or more active agents for use on skin or hair. The active agents, when combined with the polymer and optionally another solvent, serve to suspend and swell the polymer to provide an elastic, three-dimensional gel network or matrix. The active agents for the polymer include one or more liquids, a solution of a liquid and a solid dissolved in the liquid, or a mixture thereof. The active agents are under ambient conditions, and preferably have a low viscosity to provide for improved spreading on the skin.

Concentrations of the active agents in the cosmetic compositions of the present invention will vary primarily with the type and amount of polymer employed.

Active agents are selected from the group consisting of emulsifers and amphiphilic molecules, oil-soluble actives, sunscreen actives, anti-acne actives, anti-wrinkle actives, skin conditioning agents, anti-inflammatory agents, enzymatic materials, film-forming agents, and combinations thereof.

1. Emulsifiers and Amphiphilic Molecules

The compositions of the present invention may contain an emulsifier, an amphiphilic molecule, or mixtures thereof. In a preferred embodiment, the composition contains from about 0.1% to about 10% emulsifier, more preferably from about 0.5% to about 7.5%, even more preferably from about 1% to about 5%, by weight of the composition formed, of an emulsifier, amphiphilic molecule, or mixtures thereof. The emulsifier helps disperse and suspend the liquid phase, which is immiscible with the present compositions and solubilize otherwise insoluble actives in the form of micelles, vesicles, liquid crystals, lamellar phases, and other thermodynamically stable associative phases or structures. Further, the emulsifier provides the ability to coat the hair or skin surface and provides additional sensory benefits.

Known or conventional emulsifying agents can be used in the composition, provided that the selected emulsifying agent is chemically and physically compatible with essential components of the composition and provides the desired dispersion characteristics. Suitable emulsifiers include silicone emulsifiers, non-silicone-containing emulsifiers, and mixtures thereof. Suitable emulsifiers include dimethicone based emulsifying agents, emulsifying silicone elastomers, and combinations thereof. Emulsifying silicone elastomers and other silicone emulsifiers are preferred for use herein. A combination of emulsifying silicone elastomer and silicone emulsifier is also useful herein.

Silicone emulsifiers useful herein are typically organically modified organopolysiloxanes, also known to those skilled in the art as silicone surfactants. Useful silicone emulsifiers include dimethicone copolyols. These materials are polydimethyl siloxanes that have been modified to include polyether side or pendant chains such as polyethylene oxide chains, polypropylene oxide chains, mixtures of these chains, and polyether chains containing moieties derived from both ethylene oxide and propylene oxide. Also included are polydimethyl siloxanes that have been modified to include polyglycerine side chains. Other examples include alkyl-modified dimethicone copolyols, (i.e., compounds that contain C2-C30 pendant side chains). Still other useful dimethicone copolyols include materials having various cationic, anionic, amphoteric, and zwitterionic pendant moieties.

The dimethicone copolyol emulsifiers useful herein can be described by the following general structure:

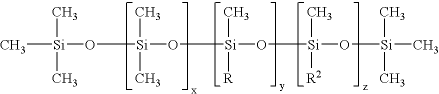

wherein R is C1-C30 straight, branched, or cyclic alkyl and R² is selected from the group consisting of

—(CH₂)ₙ—O—(CH₂CHR³O)ₘ—H,

and

—(CH₂)ₙ—O—(CH₂CHR³O)ₘ—(CH₂CHR⁴O)ₒ—H,

wherein n is an integer from 3 to about 10; R³ and R⁴ are selected from the group consisting of H and C1-C6 straight or branched chain alkyl such that R³ and R⁴ are not simultaneously the same; and m, o, x, and y are selected such that the molecule has an overall molecular weight of from about 200 to about 10,000,000, with m, o, x, and y being independently selected from integers of zero or greater such that m and o are not both simultaneously zero, and z being independently selected from integers of 1 or greater. It is recognized that positional isomers of these copolyols can be achieved. The chemical representations depicted above for the R² moieties containing the R³ and R⁴ groups are not meant to be limiting but are shown as such for convenience.

Also useful herein, although not strictly classified as dimethicone copolyols, are silicone surfactants as depicted in the structures in the previous paragraph wherein R² is:

—(CH₂)ₙ—O—R⁵,

wherein R⁵ is a cationic, anionic, amphoteric, or zwitterionic moiety, and n is as described above.

Nonlimiting examples of dimethicone copolyols also include lauryl dimethicone copolyol, dimethicone copolyol acetate, dimethicone copolyol adipate, dimethicone copolyolamine, and dimethicone copolyol behenate. See International Cosmetic Ingredient Dictionary, Fifth Edition, 1993. Examples of commercially available dimethicone copolyols useful herein sold by Dow Coming Corporation are Dow Corning® 190, 193, Q2-5220, 2501 Wax, 2-5324 fluid, and 3225C (this later material being sold as a mixture with cyclomethicone). Cetyl dimethicone copolyol is commercially available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Other nonlimiting examples include the SILWET series and SILSOFT series available from Crompton/OSi.

2. Oil-Soluble Skin Care Active

The compositions of the present invention may contain a safe and effective amount of an oil-soluble skin care active. An “oil-soluble active” may be defined as any active material that is immiscible with water. Preferably, the composition contains from about 0.001% to about 98%, by weight of the composition formed, of the oil-soluble skin care active, more preferably from about 0.01% to about 40%, even more preferably from about 0.05% to about 30%, and still more preferably from about 0.1% to about 20%, and more preferably from about 0.1% to about 10%.

Non-limiting examples of oil-soluble actives that may be used in the present invention include oil-soluble terpene alcohols, phytosterols, anti-acne actives, beta-hydroxy acids, oil soluble vitamin compounds, chelators, flavonoids, anti-inflammatory agents, anti-cellulite agents, topical anesthetics, and mixtures thereof. A preferred oil-soluble skin care active for use herein is farnesol.

a. Oil-Soluble Terpene Alcohols

As used herein, “terpene alcohol” refers to organic compounds composed of two or more 5-carbon isoprene units [CH₂═C(CH₃)—CH═CH₂] with a terminal hydroxyl group.

Examples of oil-soluble terpene alcohols that are useful herein include farnesol, derivatives of farnesol, isomers of farnesol, geraniol, derivatives of geraniol, isomers of geraniol, phytantriol, derivatives of phytantriol, isomers of phytantriol, and mixtures thereof. Preferred for use herein is farnesol.

i. Farnesol and Derivatives Thereof

Farnesol is a naturally occurring substance which is believed to act as a precursor and/or intermediate in the biosynthesis of squalene and sterols, especially cholesterol. Farnesol is also involved in protein modification and regulation (e.g., farnesylation of proteins), and there is a cell nuclear receptor which is responsive to farnesol.

Chemically, farnesol is [2E,6E]-3,7,11-trimethyl-2,6,10-dodecatrien-1-ol and as used herein “farnesol” includes isomers and tautomers of such. Farnesol is commercially available, e.g., under the names farnesol (a mixture of isomers from Dragoco) and trans-trans-farnesol (Sigma Chemical Company). A suitable derivative of farnesol is farnesyl acetate which is commercially available from Aldrich Chemical Company.

ii. Geraniol and Derivatives Thereof

Geraniol is the common name for the chemical known as 3,7-dimethyl-2,6-octadien-1-ol. As used herein, “geraniol” includes isomers and tautomers of such. Geraniol is commercially available from Aldrich Chemical Company. Suitable derivatives of geraniol include geranyl acetate, geranylgeraniol, geranyl pyrophosphate, and geranylgeranyl pyrophosphate, all of which are commercially available from Sigma Chemical Company.

iii. Phytantriol and Derivatives Thereof

Phytantriol is the common name for the chemical known as 3,7,11,15,tetramethylhexadecane-1,2,3,-triol. Phytantriol is commercially available from BASF.

b. Phytosterols

Non-limiting examples of oil-soluble phytosterol derivatives include β-sitosterol, campesterol, brassicasterol, lupenol, α-spinasterol, stigmasterol, their derivatives, and combinations thereof. Preferably, the phytosterol derivative is selected from the group consisting of β-sitosterol, campesterol, brassicasterol, stigmasterol, their derivatives, and combinations thereof.

Phytosterols are available as free sterols, acetylated derivatives, sterol esters, ethoxylated or glycosidic derivatives. More preferably, the phytosterols are free sterols. Physterols are commercially available from Aldrich Chemical Company, Sigma Chemical Company, and Dragoco.

c. Oil-Soluble Vitamin Compounds

A number of vitamins known by those in the art for providing various skin benefits are oil-soluble and some or all of their derivatives are oil-soluble. As such, these oil-soluble vitamin compounds are useful as oil-soluble skin care actives herein. Non-limiting examples of such oil-soluble vitamin compounds include retinoids, vitamin B₃ compounds, vitamin C (e.g. ascorbyl palmitate), vitamin D, vitamin K, vitamin E, and mixtures thereof. Preferred for use herein are retinoids, vitamin B₃ compounds, vitamin E compounds, and mixtures thereof, which are discussed in more detail below.

i. Retinoids

As used herein, “retinoid” includes all natural and/or synthetic analogs of Vitamin A or retinol-like compounds which possess the biological activity of Vitamin A in the skin as well as the geometric isomers and stereoisomers of these compounds. Preferred retinoids are retinol, retinyl palmitate, retinyl acetate, retinyl propionate, retinal and combinations thereof, but any oil-soluble retinoid may be used herein. These compounds are well known in the art and are commercially available from a number of sources, e.g., Sigma Chemical Company, and Boerhinger Mannheim.

ii. Oil-Soluble Vitamin B₃ Compounds

Non-limiting examples of oil-soluble B₃ compounds useful herein include nicotinic acid esters, including non-vasodilating esters of nicotinic acid (e.g., tocopheryl nicotinate). Examples of suitable vitamin B₃ compounds are well known in the art and are commercially available from a number of sources, e.g., the Sigma Chemical Company; ICN Biomedicals, Inc. and Aldrich Chemical Company.

iii. Oil-Soluble Vitamin E Compounds

Nonlimiting examples of oil soluble vitamin E compounds include tocopherol (vitamin E), tocopherol sorbate, tocopherol acetate, and other esters of tocopherol. Preferred anti-oxidants/radical scavengers are selected from tocopherol sorbate, tocopherol acetate, and mixtures thereof. Also useful herein are the class of materials, tocotrienols, which are related to vitamin E.

d. Anti-Acne Actives

Non-limiting examples of oil-soluble anti-acne actives include resorcinol and erythromycin.

e. Beta-Hydroxy Acids

Nonlimiting examples of oil-soluble beta-hydroxy acids include salicylic acid and derivatives thereof such as the octanoyl derivative. Beta-hydroxy acids are known to provide anti-acne and anti-aging benefits.

f. Chelators

As used herein, “chelator” or “chelating agent” means an active agent capable of removing a metal ion from a system by forming a complex so that the metal ion cannot readily participate in or catalyze chemical reactions. Exemplary oil-soluble chelators that are useful herein are disclosed in U.S. Pat. No. 5,487,884; International Publication No. 91/16035; and International Publication No. 91/16034. Preferred oil-soluble chelators useful in compositions of the subject invention are furildioxime, furilmonoxime, and derivatives thereof.

g. Flavonoids

Flavonoid compounds are broadly disclosed in U.S. Pat. Nos. 5,686,082 and 5,686,367. Preferred for use herein are unsubstituted flavanone, methoxy flavanones, unsubstituted chalcone, 2′,4-dihydroxy chalcone, isoflavones, and mixtures thereof. More preferred are unsubstituted flavanone, unsubstituted chalcone (especially the trans isomer), isoflavones, and mixtures thereof.

Flavonoid compounds useful herein are commercially available from a number of sources, e.g., Indofine Chemical Company, Inc., Steraloids, Inc., NovaSoy from Archer Daniels Midland Co., and Aldrich Chemical Company, Inc.

h. Anti-Inflammatory Agents

Nonlimiting examples of oil-soluble anti-inflammatory agents useful herein include steroidal anti-inflammatory agents, such as the corticosteroids (e.g. hydrocortisone), and oil soluble nonsteroidal anti-inflammatory agents, such as the propionic acid derivatives (e.g. ibuprofen, naproxen, and/or ketoprofen).

Mixtures of oil-soluble anti-inflammatory agents may also be employed, as well as the dermatologically acceptable salts and esters of these agents. For example, etofenamate, a flufenamic acid derivative, is particularly useful for topical application.

Non-limiting examples of so-called “natural” anti-inflammatory agents useful herein include those obtained as an extract by suitable physical and/or chemical isolation from natural sources (e.g., plants, fungi, by-products of microorganisms) or can be synthetically prepared. For example, candelilla wax, bisabolol (e.g., alpha bisabolol), and/or plant sterols (e.g., phytosterol), may be used.

i. Anti-Cellulite Agents

Non-limiting examples of oil-soluble anti-cellulite skin care actives useful herein include the oil soluble xanthine compounds such as caffeine.

j. Topical Anesthetics

Non-limiting examples of oil-soluble topical anesthetic drugs include benzocaine, lidocaine, and pharmaceutically acceptable salts thereof.

3. Sunscreen Actives

Compositions of the present invention may comprise a sunscreen active. Suitable sunscreens can have UVA absorbing properties, UVB absorbing properties or a mixture thereof. The exact amount of the sunscreen active will vary depending upon the desired Sun Protection Factor, i. e., the “SPF” of the composition as well as the desired level of UVA protection. The compositions of the present invention preferably comprise an SPF of at least 10, preferably at least 15. (SPF is a commonly used measure of photoprotection of a sunscreen against erythema). The SPF is defined as a ratio of the ultraviolet energy required to produce minimal erythema on protected skin to that required to produce the same minimal erythema on unprotected skin in the same individual. See, Federal Register, 43, No 166, pp. 38206-38269, Aug. 25, 1978). Compositions of the present invention preferably comprise from about 1% to about 40%, more preferably from about 4% to about 30%, by weight, of organic sunscreen. Suitable sunscreens include, but are not limited to those found in the CTFA International Cosmetic Ingredient Dictionary and Handbook, 7^{th} edition, volume 2, pp. 1672, edited by Wenninger and McEwen (The Cosmetic, Toiletry, and Fragrance Association, Inc., Washington, D.C., 1997).

The compositions of the present invention preferably comprise a UVA absorbing sunscreen active that absorbs UV radiation having a wavelength of from about 320 nm to about 400 nm. Suitable UVA absorbing sunscreen actives are selected from dibenzoylmethane derivatives, anthranilate derivatives such as methylanthranilate and homomethyl, 1-N-acetylanthranilate, oxybenzone, benzophenone-4, benzophenone-6, and mixtures thereof. Examples of dibenzoylmethane sunscreen actives are described in U.S. Pat. No. 4,387,089; and in Sunscreens: Development, Evaluation, and Regulatory Aspects edited by N. J. Lowe and N. A. Shaath, Marcel Dekker, Inc (1990). The UVA absorbing sunscreen active is preferably present in an amount to provide broad-spectrum UVA protection either independently or in combination with other UV protective actives that may be present in the composition.

The sunscreen active 4-(1,1-dimethylethyl)-4′-methoxydibenzoylmethane, which is also known as butyl methoxydibenzoylmethane or Avobenzone, is commercially available under the names of Parsol® 1789 from Givaudan Roure (International) S. A. and Eusolex® 9020 from Merck & Co., Inc. This is a solid at room temperature but can be dissolved in a solvent either present or added to the composition during the swelling process step described herein. The sunscreen 4-isoproplydibenzoylmethane, which is also known as isopropyldibenzoylmethane, is commercially available from Merck under the name of Eusolex® 8020.

The compositions of the present invention preferably further comprise a UVB sunscreen active that absorbs UV radiation having a wavelength of from about 290 nm to about 320 nm. The compositions preferably comprise an amount of the UVB sunscreen active that is safe and effective to provide UVB protection either independently or in combination with other UV protective actives that may be present in the compositions. The compositions preferably comprise from about 0.1% to about 16%, more preferably from about 0.1% to about 12%, and even more preferably from about 0.5% to about 8%, by weight, of UVB absorbing sunscreen actives.

A wide variety of UVB sunscreen actives are suitable for use herein. Nonlimiting examples of such sunscreen actives are described in U.S. Pat. Nos. 5,087,372; 5,073,371; and 5,073,372. Preferred UVB sunscreen actives are selected from 2-ethylhexyl-2-cyano-3,3-diphenylacrylate (referred to as octocrylene), cinnamates and their derivatives such as 2-ethylhexyl-p-methoxycinnamate and octyl-p-methoxycinnamate, TEA salicylate, homomenthyl salicylate, octyl salicylate, octyldimethyl PABA, camphor derivatives and their derivatives, and mixtures thereof. Preferred sunscreen actives are 2-ethylhexyl-2-cyano-3,3-diphenylacrylate (referred to as octocrylene), octyl-p-methoxycinnamate, and mixtures thereof.

An agent may also be added to any of the compositions useful in the present invention to stabilize the UV sunscreen to prevent it from photo-degrading on exposure to UV radiation and thereby maintaining its UV protection efficacy. Wide ranges of compounds have been cited as providing these stabilizing properties and should be chosen to compliment both the sunscreen and the composition as a whole. Suitable stabilizing agents include, but are not limited to, those described in U.S. Pat. Nos. 5,972,316; 5,968,485; 5,935,556; 5,827,508 and PCT patent publication WO 00/06110. Preferred examples of stabilizing agents for use in the present invention include 2-ethylhexyl-2-cyano-3,3-diphenylacrylate (referred to as octocrylene), ethyl-2-cyano-3,3-diphenylacrylate, 2-ethylhexyl-3,3-diphenylacrylate, ethyl-3,3-bis(4-methoxyphenyl)acrylate, and mixtures thereof. More preferred examples of stabilizing agents include 2-ethylhexyl-2-cyano-3 and 3-diphenylacrylate.

An agent may also be added to any of the compositions useful in the present invention to improve the skin substantivity of those compositions, particularly to enhance their resistance to being washed off by water or rubbed off. A preferred agent that will provide this benefit is a copolymer of ethylene and acrylic acid. Compositions comprising this copolymer are disclosed in U.S. Pat. No. 4,663,157.

4. Film Forming Agents

Film forming agents may be included in the compositions of the present invention to aid film substantivity and adhesion to the skin. Improving the long wear and non-transfer performance of the present compositions is desirable. Oil-soluble, silicone fluid soluble, and water insoluble film forming agents can be used in the compositions to give the desired end benefit.

Preferably, the compositions comprise from about 0.001% to about 20%, more preferably, from about 0.05% to about 10%, and even more preferably, from about 0.1% to about 5%, by weight, of the film-forming agent.

Suitable film forming agents include:

1) organic silicone resins, fluorinated silicone resins, copolymers of organic silicone resins, e.g., trimethylsiloxysilicate from GE (SR1000), GE's copolymers of silicone resins, e.g., SF1318 (silicone resin and an organic ester of isostearic acid copolymer) and CF1301 (silicone resin and alpha methyl styrene copolymer), Dow Corning's pressure sensitive adhesives—copolymers of silicone resins and various PDMS's (BIO-PSA series) and Wacker-Belsil PMS MK, Wacker-Belsil TMS 803, Wacker-Belsil SPR 45 (available from Wacker);

2) acrylic and methacrylic polymers and resins, silicone-acrylate type copolymers and fluorinated versions of, including—silicones plus polymer SA70 from 3M, KP545 from Shin-Etsu, alkyl-acrylate copolymers, e.g., KP 561 and 562 from Shin-Etsu;

3) decene/butene copolymer from Collaborative Labs;

4) polyurethanes, e.g., the Polyderm series from Alzo including but not limited to Polyderm PE/PA, Polyderm PPI-SI-WS, and Polyderm PPI-GHI; Luviset P.U.R. from BASF; Avalure UR series from Noveon;

5) styrene based materials; and

6) chitosan and chitosan based materials including cellulose and cellulose-based materials.

5. Skin Conditioning Agents

The compositions of the present invention can further comprise a skin-conditioning agent. These agents may be selected from oil-soluble humectants, exfoliants, or emollients. Preferably, the composition contains from about 0.1% to about 10%, more preferably from about 0.5% to about 7.5%, even more preferably from about 1% to about 5%, by weight of the composition formed, of a skin conditioning agent.

When the conditioning agent is an emollient it may be selected from hydrocarbons, fatty acids, fatty alcohols, and esters. Isononyl isononanoate is a preferred hydrocarbon type of emollient conditioning agent. Other hydrocarbons that may be employed include mineral oil, polyolefins such as polydecene, and paraffins such as isohexadecane (e.g. Permethyl 99 Registered TM and Permethyl 101 Registered TM). Preferably, the compositions of the present invention contain semi-solid hydrocarbons such as lanolin and lanolin derivatives, sterols (e.g., ethoxylated soya sterols), high molecular weight polybutenes and cocoa butter.

Fatty acids and alcohols will have from about 10 to about 30 carbon atoms. Illustrative of this category are pelargonic, lauric, myristic, palmitic, stearic, isostearic, hydroxystearic, oleic, linoleic, ricinoleic, arachidic, behenic and erucic acids and alcohols.

Oily ester emollients may be those selected from one or more of the following classes:

1. Triglyceride esters such as vegetable and animal fats and oils. Examples include castor oil, safflower oil, cottonseed oil, corn oil, olive oil, cod liver oil, almond oil, avocado oil, palm oil, sesame oil, squalene, Kikui oil, and soybean oil.

2. Acetoglyceride esters, such as acetylated monoglycerides.

3. Ethoxylated glycerides, such as ethoxylated glyceryl monostearate.

4. Alkyl esters of fatty acids having from about 10 to about 20 carbon atoms. Methyl, isopropyl, and butyl esters of fatty acids are useful herein. Examples include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate.

5. Alkenyl esters of fatty acids having from about 10 to about 20 carbon atoms. Examples thereof include oleyl myristate, oleyl stearate, and oleyl oleate.

6. Ether-esters such as fatty acid esters of ethoxylated fatty alcohols.

7. Polyhydric alcohol esters. Ethylene glycol mono and di-fatty acid esters, diethylene glycol mono-and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol polyfatty esters, ethoxylated glyceryl monostearate, 1,2-butylene glycol monostearate, 1,2-butylene glycol distearate, polyoxyethylene polyol fatty acid ester, sorbitan fatty acid esters, and polyoxyethylene sorbitan fatty acid esters are satisfactory polyhydric alcohol esters.

8. Wax esters such as beeswax, spermaceti, myristyl myristate, stearyl stearate.

9. C1-C30 mono- and poly-esters of sugars and related materials.

C. Solvent

The compositions of the present invention may comprise a solvent for the polymer described above. The solvent, when combined with the polymer, serves to suspend and swell the polymer to provide an elastic, three-dimensional gel network or matrix. The solvent for the polymer is liquid under ambient conditions and preferably has a low viscosity to provide for improved spreading on the skin.

Concentrations of the solvent in the cosmetic compositions of the present invention will vary primarily with the type and amount of polymer employed. Preferred concentrations of the solvent are from about 1% to about 98%, preferably from about 10% to about 60%, more preferably from about 15% to about 40%, by weight of the composition.

The solvent for the polymer comprises one or more liquid carriers suitable for topical application to mammalian skin or hair. These liquid carriers may be organic, silicone-containing or fluorine-containing, volatile or non-volatile, polar or non-polar, provided that the liquid carrier forms a three-dimensional gel network when combined with the polymer at a temperature of from about 20° C. to about 250° C., preferably from about 25° C. to about 100° C., preferably from about 28° C. to about 78° C. The solvent for the crosslinked siloxane polymer gel network preferably has a solubility parameter of from about 3 to about 13 (cal/cm³)^{0.5}, more preferably from about 4 to about 11 (cal/cm³)^{0.5}, even more preferably from about 5 to about 9 (cal/cm³)^{0.5} . Solubility parameters for the liquid carriers or other materials, and means for determining such parameters, are well known in the chemical arts. A description of solubility parameters and means for determining them are described by C. D. Vaughan, “Solubility Effects in Product, Package, Penetration and Preservation” 103 Cosmetics and Toiletries 47-69, October 1988; and C. D. Vaughan, “Using Solubility Parameters in Cosmetics Formulation”, 36 J. Soc. Cosmetic Chemists 319-333, September/October, 1988.

The solvent preferably includes volatile, non-polar oils; non-volatile, relatively polar oils; non-volatile, non-polar oils; and non-volatile paraffinic hydrocarbon oils. The term “non-volatile” as used herein refers to materials which exhibit a vapor pressure of no more than about 0.2 mm Hg at 25° C. at one atmosphere and/or to materials that have a boiling point at one atmosphere of at least about 300° C. The term “volatile” as used herein refers to all materials that are not “non-volatile” as previously defined herein. The phrase “relatively polar” as used herein means more polar than another material in terms of solubility parameter; i.e., the higher the solubility parameter the more polar the liquid. The term “non-polar” typically means that the material has a solubility parameter below about 8.0 (cal/cm³)^{0.5}.

Non-polar, volatile oils particularly useful in the present invention are selected from the group consisting of silicone oils, hydrocarbons, and mixtures thereof. Such non-polar, volatile oils are disclosed, for example, in Cosmetics, Science, and Technology, Vol. 1, 27-104 edited by Balsam and Sagarin, 1972. Examples of preferred non-polar, volatile hydrocarbons include polydecanes such as isododecane and isodecane (e.g., Permethyl-99A which is available from Presperse Inc.) and the C7-C8 through C12-C15 isoparaffins (such as the Isopar Series available from Exxon Chemicals). Non-polar, volatile liquid silicone oils are disclosed in U.S. Pat. No. 4,781,917. Particularly preferred volatile silicone oils are selected from the group consisting of cyclic volatile silicones corresponding to the formula:

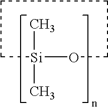

wherein n is from about 3 to about 7; and linear volatile silicones corresponding to the formula:

(CH₃)₃ Si—O—[Si(CH₃)₂—O]ₘ—Si(CH₃)₃

wherein m is from about 1 to about 7. Linear volatile silicones generally have a viscosity of less than about 5 centistokes at 25° C., whereas the cyclic silicones have viscosities of less than about 10 centistokes at 25° C. Preferred examples of volatile silicone oils include dimethicones or cyclomethicones of varying viscosities, e.g., Dow Corning 200, Dow Corning 244, Dow Corning 245, Dow Corning 344, and Dow Corning 345, (commercially available from Dow Corning Corp.); SF-1204 and SF-1202 Silicone Fluids (commercially available from G.E. Silicones), GE 7207 and 7158 (commercially available from General Electric Co.); and SWS-03314 (commercially available from SWS Silicones Corp.).

Relatively polar, non-volatile oils potentially useful in the present invention are disclosed, for example, in Cosmetics, Science, and Technology, Vol. 1, 27-104 edited by Balsam and Sagarin, 1972; U.S. Pat. No. 4,202,879 and U.S. Pat. No. 4,816,261. Relatively polar, non-volatile oils useful in the present invention are preferably selected from the group consisting of silicone oils; hydrocarbon oils; fatty alcohols; fatty acids; esters of mono and dibasic carboxylic acids with mono and polyhydric alcohols; polyoxyethylenes; polyoxypropylenes; mixtures of polyoxyethylene and polyoxypropylene ethers of fatty alcohols; and mixtures thereof. Preferred relatively polar, non-volatile solvents are branched-chain aliphatic fatty alcohols having from about 12 to about 26 carbon atoms. Also preferred relatively polar, non-volatile solvents are isocetyl alcohol, octyldecanol, octyldodecanol and undecylpentadecanol; and even more preferred is octyldodecanol. Such preferred aliphatic fatty alcohols are particularly useful in combination with the volatile liquid silicone oils discussed herein to adjust the average solubility of the solvent.

Typical non-volatile, non-polar emollients are disclosed, for example, in Cosmetics, Science, and Technology, Vol. 1, 27-104 edited by Balsam and Sagarin, 1972; U.S. Pat. Nos. 4,202,879 and 4,816,261. The non-volatile oils useful in the present invention are essentially non-volatile polysiloxanes, paraffinic hydrocarbon oils, and mixtures thereof. The polysiloxanes useful in the present invention are selected from the group consisting of polyalkylsiloxanes, polyarylsiloxanes, polyalkylarylsiloxanes, poly-ethersiloxane copolymers, and mixtures thereof, exemplified by the Dow Corning 200 fluid series (sold by Dow Corning Corp.), SF 1075 methyl-phenyl fluid (sold by General Electric Company) and 556 Cosmetic Grade Fluid (sold by Dow Corning Corp.). Non-volatile paraffinic hydrocarbon oils useful in the present invention include mineral oils and certain branched-chain hydrocarbons such as Permethyl 102A, Permethyl 103A, and Permethyl 104A (sold by Permethyl Corporation).

Additional solvents useful herein are described in U.S. Pat. No. 5,750,096.

D. Optional Ingredients

1. Solid Particles

The compositions of the present invention may further comprise one or more solid particles. The particles may be added to the end product composition, or they may be encapsulated within the polymer during polymerization of the polymer, described herein as the crosslinking of the polymer. As used herein “encapsulated” means permanent inclusion of the solid particle within the interstices (or spaces) of the carrier matrix which is the polymer. Typically, the compositions of the present invention shall include solid particles that are selected from the group consisting of shine control agents, soft focus powders, sunscreen powders, colorants, filler powders, and combinations thereof. As used herein, “colorants” generally refer to a pigment, lake, toner, dye or other agent used to impart a color expression to a material.

There are no specific limitations as to the pigment, colorant or filler powders that can be used as the “solid particle” of the composition. Each may be a body pigment, inorganic white pigment, inorganic colored pigment, pearling agent, and the like. Specific examples are talc, mica, magnesium carbonate, calcium carbonate, magnesium silicate, aluminum magnesium silicate, silica, titanium dioxide, zinc oxide, red iron oxide, yellow iron oxide, black iron oxide, ultramarine, polyethylene powder, methacrylate powder, polystyrene powder, silk powder, crystalline cellulose, starch, titanated mica, iron oxide titanated mica, bismuth oxychloride, and the like. Colorants, pigments, and powders useful herein are described in U.S. Pat. No. 5,505,937 and U.S. Pat. No. 5,688,831. Calcium and barium lakes are also used herein.

Also useful herein as solid particles are pigment and/or dye encapsulates such nanocolorants from BASF and multi-layer interference pigments such as Sicopearls from BASF.

Dispersants may also be used in conjunction with the colors and pigments of the present invention. Examples of suitable dispersants include, but are not limited to, those described in U.S. Pat. No. 5,688,493.

Certain shine control agents are suitable for use as solid particles in the present compositions. Those agents or particles include, but are not limited to, silicas, magnesium aluminum silicates, talc, sericite and various organic copolymers. Particularly effective shine control agents include silicates or carbonates that are formed by reaction of a carbonate or silicate with the alkali (IA) metals, alkaline earth (IIA) metals, or transition metals, and silicas (silicon dioxide). Preferred shine control agents are selected from the group consisting of calcium silicates, amorphous silicas, calcium carbonates, magnesium carbonates, zinc carbonates, and combinations thereof. Some specific examples of the silicates and carbonates useful in this present invention are more fully explained in Van Nostrand Reinhold's Encyclopedia of Chemistry, 4^{th} Ed. Pp. 155, 169, 556, and 849 (1984). Synthetic versions of the shine control agents, particularly silicates, are preferred. Examples of synthetic silicates useful in the present invention are Hubersorb 250® or Hubersorb 600®, available from J. M. Huber. Starch-based materials may also be used as shine control agents. Useful examples are Natrosorb W and Natrosorb HFW, DryFlo plus and DryFlo AF pure from National Starch and Chemical Company.

2. Solidifying Agent

The cosmetic compositions of this invention can contain one or more materials, herein singly or collectively referred to as a “solidifying agent”, that are effective to solidify the particular liquid base materials to be used in a cosmetic composition. (As used herein, the term “solidify” refers to the physical and/or chemical alteration of the liquid base material so as to form a solid or semi-solid at ambient conditions, i.e., to form a final composition that has a stable physical structure and is deposited on the skin during normal use conditions.)

Suitable solidifying agents include waxy materials such as candelilla, carnauba waxes, beeswax, spermaceti, carnauba, baysberry, montan, ozokerite, ceresin, paraffin, synthetic waxes such as Fisher-Tropsch waxes, silicone waxes (e.g., DC 2503 from Dow Corning), microcrystalline waxes and the like; soaps, such as the sodium and potassium salts of higher fatty acids, i.e., acids having from about 12 to about 22 carbon atoms; amides of higher fatty acids; higher fatty acid amides of alkylolamines; dibenzaldehyde-monosorbitol acetals; alkali metal and alkaline earth metal salts of the acetates, propionates and lactates; and mixtures thereof.

Hydrophobically modified celluloses are also suitable for use herein. These celluloses are described in detail in U.S. Pat. Nos. 4,228,277 and 5,104,646. Additional examples of suitable gelling agents or gellants can be found in the Cosmetic Bench Reference, p. 1.27.

3. Preservatives

Suitable traditional preservatives for compositions of this invention are alkyl esters of para-hydroxybenzoic acid. Other preservatives that have more recently come into use include hydantoin derivatives such as 1,3-bis(hydroxymethyl)-5,5-dimethylhydantoin, propionate salts, and a variety of quaternary ammonium compounds such as benzalkonium chloride, quaternium 15 (Dowicil 200), benzethonium Chloride, and methylbenzethonium chloride. Particularly preferred preservatives are disodium EDTA, phenoxyethanol, methyl paraben, propyl paraben, imidazolidinyl urea (commercially available as Germall 1157), sodium dehydroacetate and benzyl alcohol.

4. Skin Bleaching and Skin Lightening Agents

The compositions of the present invention may comprise a skin bleaching or skin lightening agent. Preferably the skin bleaching or skin lightening agents are oil-soluble. Suitable skin bleaching or skin lightening agents include those known in the art, including retinyl propionate, n-alkyl glucosamine as described in U.S. patent application Ser. No. 20020182237 A1, corresponding to PCT Application No. WO 02076423 A3, hexamidine, hydroquinone, kojic acid, arbutin, tranexamic acid. Other skin lightening materials suitable for use herein include undecylenoyl phenylalanine (Sepiwhite® from SEPPIC), aloesin, Actiwhite® (Cognis), Emblica®, and Azeloglicina. Skin lightening agents suitable for use herein also include those described in U.S. Pat. No. 6,068,834.

5. Other Optional Ingredients

A variety of additional ingredients can be incorporated into the compositions of the present invention. Nonlimiting examples of additional ingredients include additional skin care actives such as peptides, glycerol, urea, guanidine; vitamins such ascorbic acid, vitamin A, vitamin E, vitamin B₃, vitamin B₅, and mixtures thereof; analgesics; anti-histamines; and aminosilicones (e.g. General Electric/FM Series, Wacker/BELSIL Series, Osi/SILSOFT Series, and those listed CTFA Volume 1, page 107 of 9^{th} edition); anti-acne medicaments; antioxidants; anti-bacterial actives, anti-viral actives, anti-fungal actives, pore-reducing actives, and anti-perspirants; skin soothing and healing agents (e.g., aloe vera extract, allantoin); chelators and sequestrants; and aesthetic agents such as essential oils, fragrances, skin sensates, opacifiers, aromatic compounds (e.g., clove oil, menthol, camphor, eucalyptus oil, and eugenol).

II. Formulation Process

A. Crosslinking of the Polymer

The process of crosslinking the polymer is described in U.S. Pat. No. 6,531,540 B1; U.S. Pat. No. 6,444,745 B1; U.S. Pat. No. 6,346,583 B1; U.S. Pat. No. 4,970,252; U.S. Pat. No. 4,987,169; U.S. Pat. No. 5,654,326. Specifically, in the instances when crosslinked organopolysiloxane polymer gel networks are employed as polymers in the present compositions, such polymer gel networks may be derived in a number of ways, (i.e., formed from various starting materials). Suitable polymer gel networks include addition reaction-curing organopolysiloxane compositions which cure under platinum metal catalysis by the addition reaction between SiH-containing diorganopolysiloxane and organopolysiloxane having silicone-bonded vinyl groups; condensation-curing organopolysiloxane compositions which cure in the presence of an organotin compound by a dehydrogenation reaction between hydroxyl-terminated diorganopolysiloxane and SiH-containing diorganopolysiloxane; condensation-curing organopolysiloxane compositions which cure in the presence of an organotin compound or a titanate ester, by a condensation reaction between a hydroxyl-terminated diorganopolysiloxane and a hydrolyzable organosilane (this condensation reaction is exemplified by dehydration, alcohol-liberating, oxime-liberating, amine-liberating, amide-liberating, carboxyl-liberating, and ketone-liberating reactions); peroxide-curing organopolysiloxane compositions which thermally cure in the presence of an organoperoxide catalyst; and organopolysiloxane compositions which are cured by high-energy radiation, such as by gamma-rays, ultraviolet radiation, or electron beams.

Addition reaction-curing organopolysiloxane compositions are preferred for their rapid curing rates and excellent uniformity of curing. A particularly preferred addition reaction-curing organopolysiloxane composition is prepared from:

(A) an organopolysiloxane having at least 2 lower alkenyl groups in each molecule;

(B) an organopolysiloxane having at least 2 silicone-bonded hydrogen atoms in each molecule;

(C) a platinum-type catalyst; and optionally

(D) an inert liquid

With regard to the above, component (A) is the basic component of the silicone polymer gel network-generating organopolysiloxane, and curing proceeds by the addition reaction of this component with component (B) under catalysis by component (C). Component (A) contains at least 2 silicone-bonded lower alkenyl groups in each molecule; an excellent cured product will not be obtained at fewer than two lower alkenyl groups because a network structure will not be formed. Lower alkenyl groups are exemplified by vinyl, allyl, and propenyl. While the lower alkenyl groups can be present at any position in the molecule, their presence at the molecular terminals is preferred. The molecular structure of this component may be straight chain, branched straight chain, cyclic, or network, but a straight chain, possibly slightly branched, is preferred. The molecular weight of the component is not specifically restricted, and thus the viscosity may range from low viscosity liquids to very high viscosity gums. In order for the cured product to be obtained in the form of the rubbery polymer gel network, it is preferred that the viscosity at 25° C. be at least 100 centistokes.

Component (B) is an organopolysiloxane having at least 2 silicone-bonded hydrogen atoms in each molecule and is a crosslinker for component (A). Curing proceeds by the addition reaction of the silicone-bonded hydrogen atoms in this component with the lower alkenyl groups in component (A) under catalysis by component (C). Component (B) contains at least 2 silicone-bonded hydrogen atoms in each molecule in order to function as a crosslinker. Furthermore, the sum of the number of alkenyl groups in each molecule of component (A) and the number of silicone-bonded hydrogen atoms in each molecule of component (B) is to be at least 5. Values below 5 should be avoided because a network structure is then essentially not formed.

Component (C) is a catalyst of the addition reaction of silicone-bonded hydrogen atoms and alkenyl groups, and is exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum. This component is added preferably at 0.1 to 1,000 weight parts, and more preferably at 1 to 100 weight parts, as platinum-type metal proper per 1,000,000 weight parts of the total quantity of components (A) plus (B).

Component (D) is a liquid added to facilitate the reaction rate of the process if necessary.

An example of the production of the organopolysiloxane polymer gel network powder includes the process in which an organopolysiloxane composition as described above (additional-curable, condensation-curable, or peroxide-curable) is mixed with water in the presence of a surfactant (nonionic, anionic, cationic, or amphoteric). After mixing to homogeneity in a homomixer, colloid mill, homogenizer, propeller mixer, etc., this is cured by discharge into hot water (temperature at least 50° C.) and is then dried. The organopolysiloxane composition (addition-curable, condensation-curable, or peroxide-curable) is cured by spraying it directly into a heated current. The powder is obtained by curing a radiation-curable organopolysiloxane composition by spraying it under high energy radiation. The organopolysiloxane composition (addition-curable, condensation-curable, peroxide-curable) or high energy-curable organopolysiloxane composition is cured, the latter by high energy radiation, and the product is then pulverized using a known pulverizer such as, for example, a ball mill, atomizer, kneader, roll mill, etc., to thereby form the powder. Suitable organopolysiloxane polymer network powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200, and hybrid silicone powders that contain a phenyl group such as Shin-Etsu's KSP-300; and Dow Corning's DC 9506.

Preferred organopolysiloxane compositions are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer] and KSG-21 [dimethicone copolyol crosspolymer]), Grant Industries (Gransil™ line of materials), lauryl dimethicone/vinyl dimethicone crosspolymers supplied by Shin Etsu (e.g., KSG-41, KSG-42, KSG-43, and KSG-44), lauryl dimethicone/dimethicone copolyol crosspolymers also supplied by Shin-Etsu (e.g., KSG-3 1, KSG-32, KSG-33, and KSG-34). Additional polymers from Shin-Etsu which are suitable for use in the present invention include KSG-210, -310, -320, -330, and -340. Crosslinked organopolysiloxane polymer gel networks useful in the present invention and processes for making them are further described in U.S. Pat. No. 4,970,252; U.S. Pat. No. 5,760,116; U.S. Pat. No. 5,654,362; and Japanese Patent Application JP 61-18708.

Another silicone-based polymer that is suitable for inclusion into the presently claimed compositions is a polyethersiloxane block copolymer network comprising one or more polyether blocks, each comprising i) two or more structural units of the formula —R¹O— wherein each R¹ is independently a divalent hydrocarbon radical or R², wherein R² is a trivalent hydrocarbon radical, and ii) one or more polysiloxane blocks, each comprising two or more structural units of the formula —R³₂ SiO_{2/2}— wherein each R³ is independently a monovalent hydrocarbon radical or R², and wherein at least one polyether block of the copolymer network is bonded to at least one polysiloxane block of the copolymer network by a link according to formula

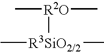

wherein the R2O unit of this formula is a unit of the at least one polyether block and the R²R³SiO_{2/2} unit of the structure of this formula is a unit of the at least one polysiloxane unit. This copolymer network is described in further detail in U.S. Pat. No. 6,444,745 B1 and U.S. Pat. No. 6,531,540.

The compositions of the present invention comprise an emulsifying, non-emulsifying, or a combination thereof, crosslinked organopolysiloxane polymer gel networks as the polymer. Emulsifying crosslinked organopolysiloxane polymer gel network can notably be chosen from the crosslinked polymers described in U.S. Pat. No. 5,412,004, U.S. Pat. No. 5,837,793, and U.S. Pat. No. 5,811,487.

B. Swelling, Gel Particle Formation, and Active Agent Entrapment

Upon completion of the crosslinking reaction steps described above in the formation of a three-dimensional organopolysiloxane network, the manufacturing process of an organopolysiloxane gel involves the intimate mixing and swelling of the polymer by one or more active agents, where the active agent becomes entrapped in the gel. Optionally, the intimate mixing and swelling of the polymer by one or more active agents in combination with one or more solvents can be employed to form a gel with entrapped active agents. This is achieved by treating the different possible combinations of polymer with active agents and optionally solvents as described above to shearing forces. To someone skilled in the art, this process is known as “kneading” or “homogenization” of the gel.

During the mixing, swelling, and gel formation phase, one or more active agents or one or more active agents in combination with one or more solvents are introduced either at once or in small increments to the now crosslinked polymer before or during the kneading process as described above. In addition, in order to facilitate an intimate mixing and swelling in formation of a gel, the input of mechanical energy in the form of shearing forces also assists the fragmentation of the gel into a desired gel particle size. The crosslinked organopolysiloxane polymer gel networks of the present invention are preferably further processed by a number of processes that introduce mechanical energy into the system to facilitate swelling and formation of the gel into gel particles of a desirable size. Examples of this type of shearing force treatments are a three-roll mill, a two-roll mill, a sand grinder, a colloid mill and a Gaulin homogenizer. Depending on the equipment used and the desired gel properties, such as particle size and gel consistency, different residence times or more than one pass of the gel system through the equipment may be necessary. One specific example of a process utilized in the present invention is subjecting the gel network to a high shear (approximately 5,000 psi) treatment in the presence of a solvent for the siloxane polymer gel network via a Sonolator at less than 10 passes. Sonolation achieves a resultant composition with polymer gel network average particle size ranging from about 5 microns to about 200 microns, preferably from about 20 to about 100 microns, more preferably from about 25 to about 80 microns and even more preferably from about 30 microns to about 65 microns as measured by the Horiba LA-910. When the compositions of the present invention are utilized for atypical cosmetic purposes, e.g., costume makeup or fashion-type makeup products, a wider particle size range would most likely be suitable. In this case, the particle of the polymers may range from about 20 microns to about 200 microns, preferably from about 30 to about 150 microns, more preferably from about 40 to about 95 microns and even more preferably from about 50 microns to about 90 microns as measured by the Horiba LA-910. As used herein, the term “particle size” of the polymer gel network represents the polymer gel network particle size in its swelled state. “Swelled,” as used herein, means that the polymer gel network particles have extended beyond their normal size and shape by virtue of their absorption of the solvent compound. Viscosity of the gel is best when ranging from about 20,000 to about 6,000,000, preferably from about 25,000 to about 4,000,000, more preferably from about 30,000 to about 3,000,000, even more preferably from about 40,000 to about 2,000,000, even more preferably from about 60,000 to about 1,500,000 cps at 25° C. as measured by a Brookfield LV Viscometer (size 4 bar, 60 rpm, 0.3 sec).

Preferably, the crosslinked organopolysiloxane polymer gel networks do not undergo recycled processing. Without being limited by theory, recycled processing produces broad particle size distributions comprising particles larger or smaller than that necessary to achieve the skin feel benefits of the present invention. Specifically, gel balls often result from silicone polymer gel network particles larger than 200 microns while polymer gel network particles smaller than 10 microns reduce skin feel and viscosity benefits. Such particle size distributions result from a failure to ensure that all of the polymer gel network particle materials experience the same shear throughout the process. Typically, with recycling, only a portion of the particles experience shear before these sheared particles are returned to the process starting point and combined with the remaining un-sheared particles. Similarly, the next cycle begins with only a portion of this particle mixture experiencing shearing before the newly sheared mixture particles are returned to the process starting point and combined with the remaining un-sheared particle mixture. Importantly, even after considerable recycling, some of the particles never actually experience shear while others experience a high degree of shear. The result is a particle size range, which encompasses particles both larger and smaller than those necessary to achieve the present invention.

In contrast, discrete pass processing, as alluded to above, ensures that all the particles experience shear as well as the same amount of shear with each run or pass. More specifically, no run or pass is completed until all the particles have experienced the same shear force. Consequently, the particle size distribution is narrower than that produced by “recycling” with respect to specific particle sizes. This results in a better balance between gel ball formation and viscosity as well as skin feel and viscosity.

### Associated Methods

Applicants have found that the compositions of the present invention are useful in a variety of applications directed to enhancement of mammalian skin and hair. The methods of use for the compositions disclosed and claimed herein include, but are not limited to: 1) methods of increasing the substantivity of a cosmetic to skin; 2) methods of moisturizing skin; 3) methods of improving the natural appearance of skin; 4) methods of applying a color cosmetic to skin; 5) methods of preventing, retarding, and/or treating wrinkles; 6) methods of providing UV protection to skin; 7) methods of preventing, retarding, and/or controlling the appearance of oil; 8) methods of modifying the feel and texture of skin; 9) methods of providing even skin tone; 10) methods of preventing, retarding, and/or treating the appearance of spider vessels and varicose veins; 11) methods of masking the appearance of vellus hair on skin; 12) methods of concealing blemishes and/or imperfections in human skin, including acne, age spots, freckles, moles, scars, under eye circles, birth marks, post-inflammatory hyperpigmentation; 13) methods of enhancing or modifying skin color such as lightening, darkening, making more pink, making more yellow, making less dull, making less ashy, making less orange, making more radiant; 14) methods of artificial tanning; 15) methods of concealing vitiligo; 16) methods of concealing damage incurred to the skin as a result of trauma, e.g., cosmetic surgery, burns, stretching of skin, etc.; and 17) methods of concealing wrinkles, fine lines, pores, uneven skin surfaces, etc. Each of the methods discussed herein involve topical application of the claimed compositions to skin.

The following examples will more fully illustrate the embodiments of this invention. All parts, percentages and proportions referred to herein and in the appended claims are by weight unless otherwise indicated.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

### Example I

A lipstick composition of the present invention is prepared as follows:

| Ingredient | Wt. % |
|---|---|
| Carnauba | 1.50 |
| Ozokerite | 5.50 |
| Candelilla | 4.00 |
| Hydrogenated Vegetable Oil | 8.50 |
| Acetylated Lanolin | 4.00 |
| Propylparaben | 0.10 |
| Cetyl Ricinoleate | 10.00 |
| Ascorbyl Palmitate | 1.00 |
| Polybutene | 2.00 |
| Polysiloxane Copolymer¹ | 5.97 |
| Stearyl Dimethicone (DC 2503 Cosmetic wax) | 5.97 |
| Anhydrous Lanolin | 5.97 |
| Elastomer-Copolyol gel blend GE 1120-21-381² | 22.95 |
| Association Structure Phase | |
| Lecithin | 5.00 |
| Niacinamide | 2.50 |
| Panthenol | 1.00 |
| Glycerine | 4.04 |
| Pigment | 9.00 |
| Water | 6.00 |

¹#1154-141-1, supplied by GE Silicones.
²Available from the General Electric Company as 12.5% silicone crosspolymer epoxy gel, 5% silicone polyether and 82.5% cyclomethicone D5.

The ingredients for the Association Structure Phase, except for the pigments, are mixed until association structures are formed. Once the association structures are formed, the pigments are added and milled on a three-roll mill. The mixture is then mixed with the remaining ingredients and mixed until a homogeneous mixture is achieved. Alternatively, the above components are added and mixed together at the same time. This mixture, with mixing, is heated to 85° C. and then poured into a mold at room temperature.

The lipstick is applied to the lips to provide color, moisturization and improved lip feel.

### Example II

A mascara of the present invention is prepared as follows:

| Ingredient | Wt. % |
|---|---|
| Carnauba Wax | 3.00 |
| Glyceryl Monostearate¹ | 7.50 |
| White Beeswax | 3.75 |
| C18-C36 Triglycerides² | 5.50 |
| Hydrogenated Glycerol Rosinate³ | 0.15 |
| Propylparaben | 0.10 |
| Paraffin Wax 118/125 | 2.25 |
| Paraffin Wax | 2.25 |
| Silicone Polyether Polymer Network⁴ | 10.00 |
| Elastomer-Copolyol gel blend GE 1120-21-381⁵ | 7.31 |
| Lecithin⁶ | 2.25 |
| Stearic Acid 3X | 4.00 |
| Oleic Acid | 0.75 |
| Triethanolamine | 1.25 |
| Potassium Cetyl Phosphate⁷ | 1.00 |
| Shellac, NF | 3.00 |
| Triethanolamine | 0.47 |
| Trisodium EDTA | 0.10 |
| Black Iron Oxide | 7.00 |
| Simethicone | 0.20 |
| Methylparaben | 0.20 |
| Ethylparaben | 0.15 |
| Phenoxyethanol | 0.80 |
| Ethyl Alcohol 40B, 190 proof | 4.00 |
| Diazolidinyl Urea | 0.20 |
| Deionized Water | 30.22 |
| dl-Panthenol | 0.35 |
| Niacinamide | 2.25 |
| Total | 100.00 |

¹Available as Emerest 2400 available from Henkel/Emery
²Available as Syncrowax HGL-C available from Croda, Inc.
³Available as Foral 105 available from Hercules, Inc.
⁴Available as Velvesil from the General Electric Company
⁵Available from the General Electric Company as 12.5% silicone crosspolymer epoxy gel, 5% silicone polyether and 82.5% cyclomethicone D5.
⁶Available as Centrolex F available from Central Soya, Inc.
⁷Available as Amphisol K available from Givaudan

The waxes and fats are mixed in a vessel equipped with a heating source. The waxes and fats are heated and mixed at low speed using a conventional blender to liquify the mixture. The mixing is continued until the mixture is homogeneous. Pigments are added to the homogenous mixture. The mixing rate is increased to high and the pigments are mixed into the mixture for about 30-35 minutes until uniformly dispersed. The mixing is continued while adding emulsifiers.

Water is added in a second vessel equipped with a heating source, followed by the niacinamide, lecithin and any other water-dispersible components. The mixture is heated and mixed to a temperature of from about 80-95° C. Additional water is added as necessary to account for water loss.

The aqueous and lipophilic mixtures are combined and mixed using a dispersator type mixer. Mixing is continued until the mixture cools to a temperature of from about 65-70° C. Preservatives are added with mixing, allowing the mixture to cool further to about 45-47° C. Any remaining components are added with mixing. The combined mixture is cooled to a temperature above the solidification point and is then poured into suitable containers.

The mascara composition is applied to the lashes and/or eyebrows to provide softening, moisturization, and conditioning.

### Example III

A moisturizing, UV-protection lotion of the present invention is prepared as follows:

| Ingredient | Wt. % |
|---|---|
| Cyclomethicone (DC245) | 17.35 |
| Elastomer-Copolyol gel blend, GE 1120-21-381¹ | 11.33 |
| Elastomer-UV active gel blend, GE 1120-21-384² | 15 |
| DC9040 Silicone Elastomer Gel³ | 10 |
| Propylparaben | 0.20 |
| Ethylene/Acrylic Acid Copolymer microspheres⁴ | 4.00 |
| Glycerin | 25.00 |
| Water | 14.00 |
| Niacinamide | 3.00 |
| Methylparaben | 0.12 |
| Total | 100.00 |

¹Available from the General Electric Company as 12.5% silicone crosspolymer epoxy gel, 5% silicone polyether and 82.5% cyclomethicone D5.
²Available from the General Electric Company as 15% silicone crosspolymer epoxy gel, 4.6% octyl salicylate, 4.5% oxybenzone-3, 10.8% octyl methoxycinnamate, 30% isododecane and 35% cyclomethicone D5.
³Available from the Dow Corning Company as 13% Dimethicone/vinyl dimethicone cross-polymer in cyclomethicone.
⁴Flobeads EA 209 supplied by Kobo Products Inc.

In a suitable stainless steel vessel, the cyclomethicone, elastomer-copolyol gel blend, elastomer-UV active gel blend, DC9040 and propylparaben are added with mixing using conventional mixing technology and mixed until homogeneous. In a separate vessel, the niacinamide and water are mixed using conventional mixing technology until homogeneous. Glycerin, ethylene/acrylic acid copolymer microspheres and methylparaben is added to the niacinamide solution with mixing until homogeneous. Next, the niacinamide mixture is combined with the cyclomethicone mixture and mixed using conventional mixing technology until homogeneous. The combined mixture is then poured into suitable containers.

The moisturizing cosmetic lotion is applied to the face and/or body to provide softening, moisturization and conditioning.

### Example IV

A liquid foundation with UV protection of the present invention is prepared as follows:

| Ingredient | Wt. % |
|---|---|
| Cyclomethicone | 11.62 |
| Elastomer-Copolyol gel blend, GE 1120-21-381¹ | 10.70 |
| Elastomer-UV active gel blend, GE 1111-19-374² | 5.38 |
| Isononyl Isononanoate | 5.00 |
| n-Propyl-4-hydroxybenzoic Acid | 0.20 |
| Ethylene Brassylate | 0.03 |
| Titanium Dioxide | 17.8 |
| Yellow Iron Oxide | 1.70 |
| Red Iron Oxide | 0.19 |
| Black Iron Oxide | 0.11 |
| Methylparahydroxybenzoate | 0.12 |
| Glycerin | 10.00 |
| 2-amino-2-methyl-1-propanol | 0.10 |
| Water | 36.45 |
| Sucrose oleate ester | 0.60 |
| | 100.00 |

¹Available from the General Electric Company as 12.5% silicone crosspolymer epoxy gel, 5% silicone polyether and 82.5% cyclomethicone D5.
²Available from the General Electric Company as 12.5% silicone crosspolymer epoxy gel, 20% octyl methoxycinnamate and 82.5% cyclomethicone D5.

The cyclomethicone, elastomer-copolyol gel blend, elastomer-UV active gel blend, isononyl isononanoate, n-propyl-4-hydroxybenzoic acid, and ethylene brassylate are added in a suitable stainless steel vessel with mixing using conventional mixing technology and mixed until homogeneous. In a separate vessel equipped with a heat source, the sucrose oleate ester and water are heated to 50° C. and mixed using conventional mixing technology until homogeneous. The sucrose oleate ester mixture is then allowed to cool to room temperature. Once cooled, the titanium dioxide, iron oxides, methylparahydroxy benzoate, glycerin, and 2-amino-2-methyl-1-propanol are added to sucrose oleate ester mixture with mixing to form a homogeneous, pigment slurry. Next, the sucrose oleate ester mixture is combined with the cyclomethicone mixture and mixed using conventional mixing technology until homogeneous. The combined mixture is then poured into suitable containers.

The liquid foundation is applied to the face to provide softening, moisturization and conditioning.

### Examples V-XIII

Cream foundations useful for providing facial moisturizing, condition, UV protection and a reduction in the appearance in oily/shiny appearance.

| | V | VI | VII | VIII | IX | X | XI | XII | XIII |
|---|---|---|---|---|---|---|---|---|---|
| | Wt % | Wt % | Wt % | Wt % | Wt % | Wt % | Wt % | Wt % | Wt % |
| Ingredient | | | | | | | | | |
| Elastomer-UV active | 9.72 | 10 | 9.72 | 10 | 10 | 10 | 10 | 10 | 14.72 |
| gel blend, GE 1120- | | | | | | | | | |
| 21-384¹ | | | | | | | | | |
| Elastomer-Copolyol | 15.5 | 20 | 15 | 15 | 20 | 20 | 20 | 20 | 10 |
| gel blend, GE 1120- | | | | | | | | | |
| 21-381² | | | | | | | | | |
| Silicone Polyether | — | 5.72 | — | 4.72 | 5.47 | 4.72 | 4.72 | 4.72 | — |
| Polymer Network³ | | | | | | | | | |
| Cyclopentasiloxane | 1.03 | 8.25 | 4.53 | 22.75 | 23.00 | 11.03 | 9.75 | 4.03 | 7.75 |
| Isoeicosane | 5.00 | 5.00 | 5.00 | 5.00 | 5.00 | 5.00 | 5.00 | 5.00 | 5.00 |
| Vitamin E Acetate | 0.50 | 0.50 | 0.50 | 0.50 | 0.50 | 0.50 | 0.50 | 0.50 | 0.50 |
| Particulates | | | | | | | | | |
| Ethylene & Acrylic | 10.00 | 10.00 | 10.00 | 5.00 | 5.00 | 10.00 | 10.00 | 10.00 | 10.00 |
| Acid Copolymer | | | | | | | | | |
| microspheres | | | | | | | | | |
| (EA209) | | | | | | | | | |
| Silica and Titanium | 2.00 | — | — | 5.00 | 2.00 | 5.00 | 5.00 | 5.00 | 5.00 |
| Dioxide and Iron | | | | | | | | | |
| Oxides (Ronasphere | | | | | | | | | |
| LDP) | | | | | | | | | |
| Silica | 3.00 | 3.00 | 2.00 | — | — | — | — | — | — |
| Magnesium | — | 2.00 | — | — | — | — | — | — | — |
| Aluminum Silicate | | | | | | | | | |
| Allyl methacrylates | 3.00 | | 3.00 | | — | 2.00 | 2.00 | 2.00 | 2.00 |
| copolymer | | | | | | | | | |
| Acrylates copolymer | 1.00 | — | — | 2.00 | — | 3.00 | 3.00 | 3.00 | 3.00 |
| Nylon 12 | | 5.00 | 1.00 | — | 2.00 | — | — | — | — |
| Aluminum Starch | 1.00 | 2.00 | — | — | — | 1.00 | 2.00 | 3.00 | 4.00 |
| Succinate | | | | | | | | | |
| Treated powders* | 1.00 | 1.00 | 1.00 | 1.00 | 1.00 | 1.00 | 1.00 | 1.00 | 1.00 |
| Film formers | | | | | | | | | |
| Polysilicone 7 | 17.00 | — | 17.00 | — | — | — | — | — | — |
| Pressure Sensitive | — | — | — | — | 3.00 | — | — | — | — |
| Adhesive | | | | | | | | | |
| Silicone Resin | — | — | — | — | — | — | 5.00 | 5.00 | 5.00 |
| Solidifying agents | | | | | | | | | |
| Ozokerite | 2.00 | 2.00 | — | — | — | — | — | — | — |
| Stearyl Dimethicone | — | — | — | 2.00 | — | — | — | — | — |
| Water Phase | | | | | | | | | |
| Deionized Water | 15.00 | 15.00 | 15.00 | 20.00 | 15.00 | 15.00 | 15.00 | 15.00 | 15.00 |
| Glycerin | 10.00 | 10.00 | 10.00 | 10.00 | 10.00 | 10.00 | 10.00 | 10.00 | 10.00 |
| Niacinamide | 2.00 | 3.50 | 5.00 | — | | | | | |
| Panthenol | 0.50 | 1.00 | 0.50 | 1.00 | 2.00 | 1.00 | 1.00 | 1.00 | 1.00 |
| Disodium EDTA | 0.10 | 0.10 | 0.10 | 0.10 | 0.10 | 0.10 | 0.10 | 0.10 | 0.10 |
| Preservatives | | | | | | | | | |
| Methyl Paraben | 0.10 | 0.10 | 0.10 | 0.10 | 0.10 | 0.10 | 0.10 | 0.10 | 0.10 |
| Benzyl Alcohol | 0.25 | 0.25 | 0.25 | 0.25 | 0.25 | 0.25 | 0.25 | 0.25 | 0.25 |
| Propyl Paraben | 0.10 | 0.10 | 0.10 | 0.10 | 0.10 | 0.10 | 0.10 | 0.10 | 0.10 |
| Ethyl Paraben | 0.20 | 0.20 | 0.20 | 0.20 | 0.20 | 0.20 | 0.20 | 0.20 | 0.20 |
| Total | 100.00 | 100.00 | 100.00 | 100.00 | 100.00 | 100.00 | 100.00 | 100.00 | 100.00 |

*= Mixture of iron oxides and titanium dioxides
¹Available from the General Electric Company as 15% silicone crosspolymer epoxy gel, 4.6% octyl salicylate, 4.5% oxybenzone-3, 10.8% octyl methoxycinnamate, 30% isododecane and 35% cyclomethicone D5.
²Available from the General Electric Company as 12.5% silicone crosspolymer epoxy gel, 5% silicone polyether and 82.5% cyclomethicone D5.
³Available as Velvesil from the General Electric Company

The cyclomethicone, elastomer-UV active gel blend, elastomer-copolyol gel blend, silicone epoxy gel, isoeicosane, and vitamin E acetate are mixed in a suitable stainless steel vessel until homogeneous. In a separate vessel equipped with a heat source, the water phase materials are heated to 50° C. and mixed until homogeneous. Sunscreen materials, preservatives, film formers, and particulates are added to the cyclopentasiloxane batch and mixed to homogeneity. If using solidifying agents, the cyclopentasiloxane mixture is heated to 80-95° C. and the solidifying agents are added until they melt. The water phase and silicone phase are cooled to below 30° C. and mixed under high shear mixing to form an emulsion.

### Example XIV

A cosmetic composition is made as follows:

| Phase | Ingredient | Wt % |
|---|---|---|
| A | Elastomer-UV active gel blend, GE 1120-21-384¹ | 23.93 |
| A | Elastomer-Copolyol gel blend, GE 1120-21-381² | 20 |
| A | Isononyl Isononanoate | 6.16 |
| B | GLW75AMPC (74.75% TiO2, 12.5% Water, 12.5% | 10.55 |
| | Glycerin, 0.25% AMP) | |
| B | GLW45YAMP (45% Fe2O3, 28.46% Water, 26.30% | 1.68 |
| | Glycerin, 0.24% AMP) | |
| B | GLW55RAMP (55% Fe2O3, 23.25% Water, 21.50% | 0.20 |
| | Glycerin, 0.25% AMP) | |
| B | GLW60BAMP (60% Fe2O3, 21.67% Water, 19.80% | 0.08 |
| | Glycerin, 0.25% AMP) | |
| B | Polyderm PE/PA | 12.10 |
| B | AMP95 | 0.09 |
| B | Glycerine | 3.53 |
| B | Water | 21.15 |
| B | SMO (O-1570 Ryoto Sugar Ester) | 0.53 |
| | Total | 100.00 |

¹Available from the General Electric Company as 15% silicone crosspolymer epoxy gel, 4.6% octyl salicylate, 4.5% oxybenzone-3, 10.8% octyl methoxycinnamate, 30% isododecane and 35% cyclomethicone D5.
²Available from the General Electric Company as 12.5% silicone crosspolymer epoxy gel, 5% silicone polyether and 82.5% cyclomethicone D5.

Each of the ingredients in phase (B) that contains the pigments are combined and mixed at 5000 RPM for 30 minutes or until ingredients are dispersed. Each of the ingredients in the non-pigment containing phase (A) are combined and mixed at a maximum of 1300 rpm until homogeneous (for about 10-15 minutes). The water phase (B) is slowly added to the silicone phase until the emulsion is properly mixed. Once mixed, the mixture is poured into a proper container and stored for use.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

What is claimed is:

1. A personal care product comprising a polymeric gel network produced by the process of:
a. crosslinking an organopolysiloxane polymer; and
b. swelling said organopolysiloxane polymer to form said gel network by introducing liquids including one or more active agents, wherein said active agents are entrapped in said gel network.

2. The product of claim 1 wherein said liquids include one or more solvents.

3. The product of claim 1 wherein said organopolysiloxane polymer is selected from the group consisting of linear polymers, non-linear polymers, and combinations thereof.

4. The product of claim 1 wherein said organopolysixloane polymer is a crosslinked organopolysiloxane polymer gel network selected from the group consisting of non-emulsifying polymer gel networks, emulsifying polymer gel networks, and combinations thereof.

5. The product of claim 1 wherein said active agents are selected from the group consisting of emulsifers and amphiphilic molecules, oil-soluble actives, sunscreen actives, anti-acne actives, anti-wrinkle actives, anti-inflammatory actives, skin conditioning agents, enzymatic materials, film-forming agents, and combinations thereof.

6. The product of claim 5 wherein said emulsifiers are selected from the group consisting of dimethicone based emulsifying agents, emulsifying silicone elastomers, and combinations thereof.

7. The product of claim 6 wherein said emulsifier is a dimethicone copolyol.

8. The product of claim 5 wherein said oil-soluble actives are selected from the group consisting of oil-soluble terpene alcohols, phytosterols, anti-acne actives, beta-hydroxy acids, chelators, flavonoids, anti-inflammatory agents, anti-cellulite agents, topical anesthetics, and combinations thereof.

9. The product of claim 5 wherein said sunscreen actives are selected from the group consisting of 2-ethylhexyl p-methoxycinnamate, octocrylene, oxybenzone, homomenthyl salicylate, octyl salicylate, dibenzoylmethane derivatives, camphor derivatives, avobenzone, benzophenone-4, benzophenone-6, and mixtures thereof.

10. The product of claim 2 wherein said solvent is selected from the group consisting of silicone oils, hydrocarbons, fatty acids, fatty alcohols, esters, and combinations thereof.

11. The product of claim 1 wherein said personal care product is selected from the group consisting of lipsticks, lip balms, makeup, foundations, blushers, face powders, mascaras, eyeliners, eyeshadows, concealers, moisturizers, and sunscreens.
